(19) United States
(12) Patent Application Publication
(10) Pub. No.: US 2005/0232888 A1
(43) Pub. Date: Oct. 20, 2005

(54) Cosmetic compositions for hair care

(76) Inventors: Dirk Weber, Marly (CH); Maryline Kolly, Givisiez (CH)

Correspondence Address: STRIKER, STRIKER & STENBY, 103 EAST NECK ROAD, HUNTINGTON, NY, 11743, US

(21) Appl. No.: 11/103,260

(22) Filed: Apr. 11, 2005

(30) Foreign Application Priority Data
Apr. 14, 2004 (DE) 102004018017.2

(51) Int. Cl. A61K 7/06; A61K 7/11

(52) U.S. Cl. 424/70.12

(57) ABSTRACT

The cosmetic composition with hair conditioning action contains from 0.01 to 20.0 percent by weight of at least one siloxane polymer of formula (I): 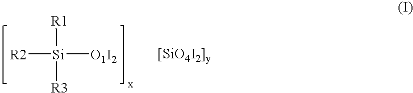
wherein R1, R2 and R3 each represent a linear or branched C₁- to C₆-alkyl group, phenyl group or a linear or branched phenyl-(C₁- to C₆)-alkyl group, wherein x and y represent whole numbers, x≧4 and y≧1 and y has a maximum value of 100,000 and x has a maximum value of 100,002; and from 0.1 to 20.0 percent by weight of at least one polyhydric alcohol. The polyhydric alcohol is preferably an aliphatic polyhydric alcohol with 2 to 4 carbon atoms. The composition optionally contains at least one cationic surfactant and/or at least one fatty alcohol. This hair conditioning agent is preferably used as a hair rinse.

### BACKGROUND OF THE INVENTION

The present invention concerns a cosmetic composition for hair care. Especially the invention concerns a cosmetic composition with hair conditioning action, which imparts a silky smooth feeling and especially softness to the hair and preferably is formed as a rinse-off composition. This cosmetic composition includes a combination of a silicon compound, a multivalent alcohol and a cationic surfactant.

Conventional hair conditioning preparations are formulated as rinse-off care or leave-on compositions generally based on aqueous emulsions. Cation-active substances, for example cationic surfactants, hydrophobic substances, such as fatty alcohols, and other oily components, emulsifiers and additional specific effective and perfume substances are essential ingredients. The most important ingredients are cationic surfactants, fatty alcohols, silicon compounds and emulsifiers. The primary objects of hair conditioning agents are the improvement of manageability, combability, the luster and feel of the treated hair. The treated hair often feels heavy and loaded, which is not always desirable. These often observed undesirable effects are very strongly dependent on the hair type. Particularly ethnic hair types are not satisfactorily treated with commercial hair conditioning agents.

Products, with which good results may be obtained concerning silky and smooth feel, manageability and softness of dry hair, especially with difficult types of hair, are desired.

Difficult or problematic hair types typically are found on Asiatic people. This full, smooth and heavy hair very easily tends to require excessive care and is difficult to set in a hair style. There are two distinctive dry types, fashion-dependent for Asiatic people: the Sara-Sara type (“flexible-dry” or “supple-dry”), for airy-light fluffy or loose hairstyles, and the Shittori type (“flexible-moist” or “supple-moist”), which is typically for optically more placid or quiet, smoother hairstyles.

Conditioning hair care agents are known in various forms. They are based on polymers with thickening action, such as cellulose derivatives (trademarks: NATROSOL®, METHOCEL®), high molecular weight Chitosan derivatives (trademark: KYTAMER® PC), complex polysaccharides (tradenames: Karaya gum, Tragacanth gum, JAQUAR® types, KELTROL® types), acrylic acid polymerizates or silicones. For example, WO 01/28506 (Wella AG) describes a water-in-silicone oil hair conditioning composition, which contains a certain silicone surfactant, a hydrophobic non-surfactant silicone compound and a hydrophobic non-surfactant polyhydric alcohol.

Such hair conditioning compositions have however the great disadvantage that their care action is so weak, especially with the above-mentioned problematic hair, that it is far from that of a classical hair care composition based on a mixture of fatty alcohol, quaternary surfactants and silicones. Especially the distributability of this product on full hair and the rinsability are poorer in comparison to a typical liquid crystal gel. Furthermore the dry hair feels clearly loaded or heavy.

### SUMMARY OF THE INVENTION

It is an object of the present invention to provide a composition, which fulfills the typical requirements regarding hair conditioning and distributability and also for typical Asiatic hair regarding silky and smooth feel, manageability and softness and with which good action may be obtained in the distinctive Shittori Effect (generally defined as “flexible-moist”).

It has now been found that this object is attained in an outstanding manner by a hair conditioning agent of the following composition.

Thus the subject matter of the invention is a cosmetic composition with conditioning action for hair containing
a) from 0.01 to 20.0 percent by weight of at least one siloxane polymer of formula (I): 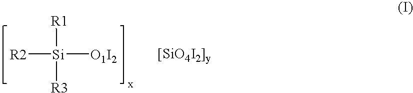
   wherein R1, R2 and R3 are each, independently of each other, a linear or branched C₁- to C₆-alkyl group, phenyl group or a linear or branched phenyl-(C₁— to C₆)-alkyl group, and wherein x and y represent whole numbers, x≧4 and y≧1, and y has a maximum value of 100,000 and x has a maximum value of 100,002; and
b) from 0.1 to 20.0 percent by weight of at least one polyhydric alcohol.

Preferably preferred embodiments of the cosmetic composition contain from 0.1 to 20.0 percent by weight of at least one fatty alcohol as additional component (c) and/or from 0.1 to 10.0 percent by weight of at least cationic surfactant as additional component (d).

The agent fulfills the requirements for a hair conditioning agent regarding conditioning action. The agent can be distributed satisfactorily on the hair. The hair is both noticeably smoother and softer after treatment both in a moist and also in a dry state. The hair is combed better and set in a hairstyle better, disentangled and lustrous.

The cosmetic composition according to the invention can thus be used preferably for improvements of the feel, luster, flexibility, manageability, combability, softness and weight (Shittori) of hair.

The siloxane polymer of component (a) is present in the agent according to the invention in an amount of 0.01 to 20.0, preferably of 0.1 to 10.0, especially preferably from 0.1 to 5.0, and most preferably from 0.5 to 3.0, percent by weight (wt. %).

Preferred siloxane polymers are those, in which R1, R2 and R3 each represent, independently of each other, a methyl, propyl, butyl, phenyl, phenyl-propyl or phenylisopropyl group, x and y represent whole numbers, x≧4 and y≧1, and y has a maximum value of 100,000 and x has a maximum value of 100,002.

Preferred siloxane polymers, which are especially preferred, include those of the following formula (I): 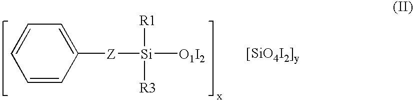
wherein Z represents a branched or unbranched C₁- to C₆-alkylene group, R1 and R2 each represent a C₁- to C₆-alkyl group, and x and y are whole numbers, x≧4 and y≧1, and y has a maximum value of 100,000 and x has a maximum value of 100,002.

Siloxane polymers of the following formula (III) are especially preferred: 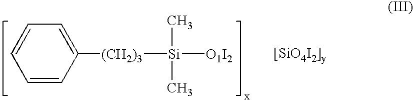
wherein x and y are whole numbers, x≧4 and y≧1 and y has a maximum value of 100,000 and x has a maximum value of 100,002.

An especially preferred siloxane polymer of this type has the INCI name phenylpropyldimethylsiloxysilicate and is commercially obtainable from GE Bayer silicones under the trademark SILSHINE® 151 and/or under the trademark BAYSILONE® CF-1301 (GE Silicones) according to the International Cosmetic Ingredient Dictionary and Handbook, Tenth Edition, 2004, Volume 2, p. 1370.

However a siloxane polymer according to formula (III) with the above-described definition, which is characterized by an index of refraction n²⁰_{D} between 1.49 and 1.52, preferably 1.51, is especially preferred. This sort of siloxane polymer has a viscosity between 280 and 600 mPas, measured at 25° C. with the Haake rotary viscometer Type VT 550 (measurement system comprising a measurement beaker or container MV-DIN and a rotating body according to DIN 53019/ISO 3219) with a shear rate of 12.9 per second.

Also at least one other silicone compound with hair conditioning properties can be added to the combination according to the invention. In principle all silicone compounds are suitable, which are insoluble in the aqueous phase and are low molecular weight, easy-to-volatilize silicones or high molecular weight, difficult-to-volatilize silicones. Non-volatile silicones are silicones with no or only slight vapor pressure under normal conditions (1 atmosphere, 25° C.). These silicones remain in the hair, after they are applied. Volatile silicones in contrast evaporate after application to the hair under normal conditions in a time interval of typically about two hours. The high molecular weight silicones can be silicone resins or silicone gum, which are soluble in a low molecular weight liquid silicone. The low molecular weight silicone oils have a viscosity of preferably 100 to 1000 mPas, and the high molecular weight silicone oils have a viscosity of above 1,000 to 2,000,000 mPas, measured at 25° C. with a Haake rotary viscometer Type VT 550 (measurement system as already described above) and a shear rate of 12.9 per second.

Hydrophobic silicones generally can include cyclic polydimethylsiloxane (INCI name: cyclomethicone), such as octamethyl cyclotetrasiloxane and decamethylcyclopentasiloxane; linear polydimethylsiloxane (INCI-name: dimethicone), polydiethylsiloxane, additional phenyl substituted siloxanes, such as those with the INCI name phenyltrimethicone, polymethylphenylsiloxane, phenylpropyltrimethicone, phenylpropyltrimethicone/diphenylmethicone, diphenyldimethicone, diphenylisopropyldimethicone, triphenyltrimethicone, hydroxysubstituted siloxane (INCI name: dimethiconol) or their mixtures.

Ingredient (b) comprises a branched or unbranched polyhydric aliphatic alcohol, preferably with at least two hydroxyl groups, which includes also sugar alcohol, preferably with 2 to 6 carbon atoms. The polyhydric aliphatic alcohols, for example, can be ethylene glycol, 1,3-butylene glycol, 1,2-propylene glycol, propan-1,3-diol, butan-1,3-diol, caprylyl glycol, glycerol or sorbitol. The polyhydric or multivalent alcohol is used in the composition according to the invention preferably in an amount of 0.1 to 50 percent by weight, especially from 1.0 to 25% by weight, particularly of 2.0 to 10.0% by weight, for example 5% by weight.

Besides the polyhydric alcohol the composition according to the invention can also contain lower monohydric or monovalent alcohols with from 1 to 4 carbon atoms, for example ethanol or isopropanol.

Preferably the composition according to the invention can contain at least one fatty alcohol as the additional ingredient (c).

The fatty alcohols that can be used in the composition according to the invention include all those fatty alcohols that can be used for cosmetic purposes. In the sense of the present invention the “fatty alcohols” are those of the formula
(IV): R²OH (IV),
wherein R² represents an aliphatic, linear or branched C₁₀- to C₂₂-alkyl group. Preferred fatty alcohols include those, in which R² represents an aliphatic linear or branched C₁₀- to C₂₂-alkyl group. For example, capryl alcohol, 2-ethylhexyl alcohol, lauryl alcohol, myristyl alcohol, oleyl alcohol, cetyl alcohol, stearyl alcohol, isostearyl alcohol, behenyl alcohol, linoleyl alcohol, linolenyl alcohol, ricinoleyl alcohol, cetearyl alcohol and mixtures thereof are suitable as the fatty alcohol. The fatty alcohol is present in the composition according to the invention in an amount of from 0.1 to 20.0% by weight, preferably 0.5 to 10.0% by weight, especially preferably from 1.0 to 8.0% by weight, for example 4.0% by weight.

Cationic surfactants preferred as the additional ingredient (d) in the composition according to the invention include those which contain quaternary ammonium groups. The additional ingredient (d) can be a cationic surfactant or an amphoteric betainic surfactant. However cationic surfactants are preferred. Suitable cationic surfactants contain amino groups or quaternarized hydrophilic ammonium groups, which have a positive charge in solution and which can be represented by the following general formula (V):
N⁺R¹R²R³R⁴X⁽⁻⁾ (V),
wherein each of R¹ to R⁴, independently of each other, denote an aliphatic group, an aromatic group, an alkoxy group, a polyoxyalkylene group, an alkylamido group, a hydroxyalkyl group, an aryl group or an alkaryl group, each with from 1 to 22 carbon atoms. X⁽⁻⁾ is a cosmetically compatible anion. The anion can be a halogen, acetate, phosphate, nitrate or alkyl sulfate anion. However chloride is preferred.

In order to guarantee surfactant properties each of the R¹ to R⁴ groups has at least eight carbon atoms. The aliphatic groups can also contain cross-linkages or other groups, for example amino groups, besides the carbon atoms and the hydrogen atoms. For example, suitable cationic surfactants include ammonium halides, especially the chlorides or bromides, such as alkyldimethylbenzyl-ammonium salts, alkyltrimethylammonium salts, dialkyldimethylammonium chlorides and trialkylmethylammonium chlorides or bromides, for example cetyltrimethylammonium chloride or bromide, stearyltrimethylammonium chloride or bromide, distearyltrimethylammonium chloride or bromide, lauryldimethyl-benzylammonium chloride or bromide, tetradecyltrimethylammonium chloride or bromide, alkyldimethylhydroxyethylammonium chlorides or bromides, alkyl-trimethylammonium chlorides or bromides, dialkyldimethylammonium chlorides or bromides, alkylpyridinium salt, especially lauryl or cetyl pyridinium chloride, alkylamidoethyltrimethylammonium ether sulfate and compounds with cationic character, such as amine oxides, especially alkylmethylamine oxides or alkylaminoethyldimethylamine oxides. Cetyltrimethylammonium chloride or cetrimonium chloride are especially preferred, which is marketed, for example, in the form of a 26% percent aqueous solution under the trademark DEHYQUART® A from Cognis and under the trademark GENAMIN® CTAC of Clariant and in the form of a 50 percent solution in isopropanol under the trademark ARQUAD® 16-50 of Akzo Nobel.

The cationic surfactant is present in the composition according to the invention in an amount of 0.1 to 10.0 wt. %, preferably 0.5 to 8.0 wt. % and especially preferably 1.0 to 5.0 wt. %, for example 2.0 wt. %.

The agent according to the invention is preferably in the form of a liquid crystal gel with a viscosity preferably in a range of 1000 to 10000 mpas, especially preferably from 2500 to 7000 mpas, measured at 25° C. with a Haake rotary viscometer Type VT 550 (measurement system comprising a measurement beaker or container MV-DIN and a rotating body according to DIN 53019/ISO 3219) with a shear rate of 12.9 per second.

The composition according to the invention preferably has a pH in an acid pH range, especially from 1.0 to 6.9 (for example 4.0). The pH can be adjusted by any suitable organic or inorganic acid, for example, by formic acid, tartaric acid, malic acid, maleic acid, fumaric acid, pyrrolidone carboxylic acid, citric acid, lactic acid, sulfuric acid, acetic acid, hydrochloric acid, phosphoric acid, glyoxylic acid, among others.

The agent according to the invention can also contain conventional cosmetic additives that are usually used in hair treatment agents, for example fixing or non-fixing nonionic, anionic, cationic, zwitterionic or amphoteric polymers and their combinations in an amount of preferably from 0.01 to 10 percent by weight; additional wetting agents or emulsifiers from the classes of anionic, cationic, amphoteric or non-ionogenic surface-active substances in an amount of preferably from 0.01 to 10 percent by weight; moisturizing agents, preservatives, bactericidal and fungicidal agents, such as 2,4,4-trichloro-2-hydroxydiphenyl ether, parabene or methylchloroisothiazolinone, in an amount of 0.01 to 1.0 percent by weight; buffer substances, such as sodium citrate or sodium phosphate, in an amount of 0.1 to 1.0 percent by weight; coloring agents, such as fluorescein sodium salt in an amount of about 0.1 to 1.0 percent by weight; hair dye compounds, in an amount of about 0.001 to 0.1 percent by weight; care ingredients, such as plant and vegetable extracts, protein and silk hydrolyzates, lanolin derivatives, in an amount of 0.1 to 5 percent by weight; light protective agents, sun screens, antioxidants, radical trapping agents, anti-flaking ingredients, luster-imparting agents, vitamins and lubricating agents in an amount of 0.01 to 10% by weight.

As hair dye compounds all physiologically compatible known direct dyeing dye compounds can be used. These direct dyeing dye compounds include especially:
Nitro dye compounds (blue) for example, 4-N-ethyl-N-(2′-hydroxyethyl)amino-1-(2″-hydroxyethyl)amino-2-nitrobenzene, 1-amino-3-methyl-4-(2′-hydroxyethyl)amino-6-nitrobenzene, 1-(2′-hydroxyethyl)amino-2-nitro-4-bis-(2″-hydroxyethyl)aminobenzene, 4-bis-(2′-hydroxyethyl)amino-1-(2″-methoxyethyl)amino-nitrobenzene, 1-(2′,3′-dihydroxypropyl)amino-2-nitro-4-[ethyl-2″-(hydroxyethyl)amino]-benzene, 1-[(2′,3′-dihydroxypropyl)amino]-2-nitro-4-[ethyl-2″-(hydroxyethyl)amino]-benzene, 1-(3′-hydroxypropylamino)-2-nitro-4-bis-(2″-hydroxyethylamino)-benzene;
Nitro dye compounds (red) for example, 1-amino-4-(2′-hydroxyethyl)-amino-nitrobenzene, 1-hydroxy-2-amino-4,6-dinitro-benzene, 1-amino-2-nitro-4-bis-(2′-hydoxyethyl)aminobenzene, 1-amino-2-nitro-4-(2′-hydoxyethyl)amino-5-chlorobenzene, 1-(2′-hydoxyethyl)amino-2-nitro-4-aminobenzene, 1-hydroxy-3-nitro-4-aminobenzene, 1-(2′-aminoethyl)amino-2-nitro-4-(2″-hydroxyethyl)-oxybenzene, 3-nitro-4-(2′-hydroxyethyl)amino-phenylglyceryl ether, 1-amino-5-chloro-4-(2′,3′-dihydroxypropyl)amino-2-nitrobenzene, 1,4-bis-[(2′,3′-dihydroxypropyl)amino-5-chloro-2-nitrobenzene, 1-hydroxy-2-(2′-hydroxyethyl)amino-4,6-nitrobenzene, 2-amino-6-chloro-4-nitropenyl, 1-hydroxy-3-nitro-4-(3′-hydroxypropylamino)-benzene, 3-nitro-4-ethylamino-benzoic acid, 4-amino-2-nitrodiphenylamino-2-carboxylic acid, 2-chloro-6-ethylamino-4-nitrophenol, 2,5-diamino-6-nitropyridine, 1,2,3,4-tetrahydro-6-nitroquinoxaline;
Nitro dye compounds (yellow) for example, 4′-(2′-hydroxyethyl)amino-3-nitrobenzonitrile, 4-(2′-hydroxyethyl)amino-3-nitrobenzamide, 1-amino-2-(2′-hydroxyethyl)amino-5-nitrobenzene, 1-methoxy-2-(2′-hydroxyethyl)amino-5-nitrobenzene, 1-hydroxy-3-nitro-4-(2′-hydroxyethyl)-amino-benzene, 1-hydroxy-2-amino-3-nitrobenzene, 1-amino-2-methyl-6-nitro-benzene, 1-(2′-hydroxyethyl)-oxy-3-methylamino-4-nitrobenzene, 1-methylamino-2-nitro-5-(2′,3′-dihydroxypropyl)-oxybenzene, 1-(2′-hydroxyethyl)-amino-2-hydroxy-4-nitrobenzene, 1-methoxy-3-(2′-aminoethyl)-amino-4-nitrobenzene, 1-(2′-ureidoethyl)-amino-4-nitrobenzene, 1-(2′-hydroxyethyl)-amino-2-nitro-benzene, 4-(2′-hydroxyethyl)-amino-3-nitrotrifluoromethylbenzene, 2,4-bis-[N-(2′-hydroxyethyl)-amino]-5-chloronitrobenzene, 4-(2′,3′-dihydroxypropyl)-amino-3-nitro-trifluoromethylbenzene, 4-(2′-hydoxymethyl)amino-3-nitro-methylbenzene, 4-(2′-hydoxyethyl)amino-3-nitrochlorobenzene;
Azo dye compounds for example, 1-(4′-nitrophenylazo)-2-methyl-4-bis-(2″-hydroxyethyl)aminobenzene, 1-(3′-nitro-4-amino)-phenylazo-2-hydroxy-7-trimethylammonium chloride naphthalene, 1-(2′-hydroxy-4′-sulfo-6′-nitro)-naphthyl-azo-2-hydroxynaphthalene, 1-(4′-amino-phenylazo)-2-methyl-4-bis-[(2′-hydroxyethyl)-amino]-benzene, 5-(4′-dimethyl-amino-phenylazo)-1,4-dimethyl-triazonium chloride, 1-(2′-methoxyphenylazo)-2-hydroxy-7-trimethylammonium naphthalene chloride, 1-(4′-aminophenylazo)-2-hydroxy-7-trimethylammonium naphthalene, 4-(3′-trimethyl-ammonium-phenylazo)-N-phenyl-3-methylpyrazolone (5), 4-hydroxy-3-[(4′-sulfo-1′-naphthyl)-azo]-1-naphthalene sulfonic acid, 1-(4′-sulfophenylazo)-2-hydroxy-naphthalene, 1-(4′-sulfophenylazo)-2-hydroxy-6-sulfo-naphthalene, 4-amino-[4′-bis-(2″-hydroxyethyl)]-amino-azobenzene, 4-amino-[4′-bis-(2″-hydroxyethyl)amino]-2′-methyl-azobenzene, 3-(2′,6′-diaminopyridyl-3′-azo)-pyridine, 7-phenylazo-1-amino-3,6-disulfo-8-hydroxy-naphthalene, 5-acetylamino-4-hydroxy-3-[(2′-methyl-phenyl)azo]-2,7-naphthalene disulfonic acid, 2-(2′,4′-dimethyl-phenylazo)-6-(4″-sulfophenylazo)-1,3-dihydroxybenzene;
Quinone dye compounds, such as, 1,4-bis-(2′,3′-dihydroxypropyl)amino-anthraquinone, 1-methylamino-4-(2′-hydroxyethyl)amino-anthraquinone, 2-(2′-aminoethyl)amino-anthraquinone, 2-bromo-4,8-diamino-6-(3′-trimethylammonium)-phenylamino-1,5-naphtha-quinone, 1-(2′-sulfo-4′-methyl-phenyl)-amino-4-hydroxy-anthraquinone, 1,4-diaminoanthraquinone, 1-amino-2-sulfo-4-cyclohexylamino-anthraquinone, 1-methylamino-4-aminopropyl-aminoanthraquinone, 1-aminopropylamino-anthraquinone;
Triphenylmethane dye compounds, for example, 4′, 4″, 4′″-triamino-3-methyl-triphenylcarbonium chloride, bis-(4,4-diethylamino-phenyl)-4′-ethylanalino-naphthyl-carbonium chloride, bis-(4,4-dimethylamino-phen)-4′-phenyl-amino-naphthyl-carbonium chloride and 4,4-bis(N-ethyl-3-sulfobenzyl)-amino-2″-sulfofuchsonium.

Also natural dye stuffs, such as henna, indigo or juglone, can be employed.

Sun protective agents or sun screens can be included in the composition according to the invention. They include all known organic and inorganic UV-filtering and/or light protective pigments. Typical UV-filtering compounds are, e.g., derivatives of benzoylmethane, for example 1-(4′-tert.butylphenyl)-3-(4′-methoxyphenyl)propan-1,3-dione, 4-tert.butyl-4′-methoxydibenzoylmethane (PARSOL® 1789), and derivatives of triazines, for example 2,4-bis-{[4-(2-ethyl-hexyloxy)-2-hydroxy]-phenyl}-6-(4-methoxyphenyl-1,3,5-triazine (TINOSORB® S), 4,4′,4″-(1,3,5-triazine-2,4,6-triyltriimino)trisbenzoate (UVINUL® T 150) or derivatives of 3,3-d iphenylacrylate, such as ethylhexyl-2-cyano-3,3-diphenyl-acrylate (octocrylene), or mixtures thereof.

Light protective pigments, which can be employed, include finely dispersed metal oxides, such as zinc oxide, titanium oxide and oxides of iron, zirconium, silicon, manganese, aluminum, cerium and their mixtures.

The composition according to the invention can be in the form of a shampoo, a cream, a lotion or a fluid gel or pourable gel, pump foam or aerosol foam.

If the composition is in a low viscosity form, it can also be sprayed in order to achieve an especially good distributability on the hair. The hair treatment composition according to the invention is then used in combination with a suitable mechanical spraying device. The term “spraying device” here means an apparatus, which permits the spraying of a liquid without using a propellant. For example, spray pumps or elastic containers provided with a spray valve, in which the cosmetic agent according to the invention has been filled under pressure, are suitable mechanical spraying devices. In operation the agent is continuously dispensed from the filled expanded elastic container when the user opens the spray valve.

The composition according to the invention is preferably used as a rinse-off product, but also as a leave-on product. In a typical application as a rinse-off product a quantity of the agent sufficient to achieve the desired conditioning effect is distributed on wet or moist hand-towel-dried hair after washing the hair. The quantity employed depends on the hair abundance and amounts to typically about 1 to 25 g, preferably 3 to 10 g. After a sufficient acting time, for example 1 to 15 minutes, the hair is rinsed. In case only a gentle conditioning effect is desired, it can be rinsed out immediately. Subsequently the hair is combed as needed or set in a hair-do and dried.

When the composition according to the invention is used as a leave-on product preferably from 0.5 to 3.0 g of the product are worked into moist or dry hair. The cuticle is smoothed and undesired curling removed. The composition imparts luster, stylability, control and structure to the hair.

The following examples illustrate the above-described invention in more detail, but the details in these examples should not be considered as limiting the claims appended hereinbelow. In the following examples % means percent by weight and relates to the total amount of the final product.

### EXAMPLES

### Example 1

### Manufacture of a Hair Conditioning Agent in the form of a Liquid Crystal Gel

Cetearyl alcohol in an amount of 4.0 g was heated together with the heat-stable oil-soluble ingredients (1.0 g paraffin oil, 0.1 g propylparabene) at 80° C. The non-heat sensitive, water-soluble ingredients (4.0 g of 50% solution of cetyltrimonium chloride—GENAMIN® CTAC 50—in isopropanol, and 0.1 g of methylparabene) were dissolved at 80° C. in the largest part of the water. Both phases were combined at 80° C., stirred for five minutes at this temperature and subsequently cooled with stirring. Besides the phenylpropyldimethylsiloxysilicate (1.5 g SILSHINE® 151) also the silicone Amodimethicone (1.0 g) and the other ingredients (4.0 g propylene glycol, 0.5 g perfume oil, 1.0 g GLUADIN® APG, 0.1 g PARSOL® 1789) are worked in and water is added to 100 g.

### Example 2

### Hair Conditioning Agent (Conditioner 1)

| | |
|---|---|
| 4.0% | Cetearyl alcohol |
| 2.0% | Cetrimonium chloride |
| 2.0% | Phenylpropyldimethylsiloxysilicate |
| | (SILSHINE ® 151) |
| 5.0% | Propylene glycol |
| 0.3% | Citric acid |
| 0.5% | Preservative (NIPASEPT ®) |
| 0.5% | Perfume |
| To 100% | Water |

This hair conditioning agent (Conditioner 1) was compared with three commercially available prior art hair conditioning agents (Conditioner 2, 3 and 4) in Table I below.

TABLE I
COMPARISON OF EXAMPLES OF THE PRIOR ART CONDITIONERS
WITH THE CONDITIONER OF THE INVENTION

| | Conditioner | Conditioner | Conditioner | Conditioner |
|---|---|---|---|---|
| Ingredient | 1 | 2 | 3 | 4 |
| Cetyl alcohol | 4.0% | 4.0% | 4.0% | 4.0% |
| Cetrimonium Chloride | 2.0% | 2.0% | 2.0% | 2.0% |
| SILSHINE ® 151 | 2.0% | — | — | — |
| Propylene Glycol | 5.0% | — | 5.0% | 5.0% |
| Dimethicone | — | 2.0% | 2.0% | — |
| Perfume | 0.5% | 0.5% | 0.5% | 0.5% |
| Citric acid | 0.3% | 0.3% | 0.3% | 0.3% |
| Water | To 100 | To 100 | To 100 | To 100 |

The evaluation of these conditioners took place according to the so-called half side comparison, in which first the entire head is washed with a shampoo and subsequently the left half side is treated with a test sample (conditioner 2, 3 or 4) and the right half side is treated with a composition according to the invention (conditioner 1). Both sides were rinsed out at the same time and subsequently dried with a hand towel. Subsequently the feel, combability and load of the hair in the moist state on both sides were compared. After that the entire head was styled and dried. The evaluation occurred in the dry state.

The feel, combability, luster and manageability of prior art conditioners 2, 3 and 4 were clearly inferior to that of conditioner 1 according to the invention in this test, especially for Asiatic hair. Furthermore the characteristic properties of conditioner 1 are clearly within a range necessary for the Shittori style.

### Example 3

### Hair Conditioning Agent (Hair Care)

| | |
|---|---|
| 6.0% | Cetearyl alcohol (LANETTE ® 16) |
| 2.0% | Cetrimonium chloride (GENAMIN ® CTAC 50) |
| 1.0% | Behentrimonium chloride (GENAMIN ® KDMP) |
| 1.5% | Phenylpropyldimethylsiloxysilicate |
| | (SILSHINE ® 151) |
| 0.5% | Dimethicone |
| 2.5% | Butylene glycol |
| 0.3% | Citric acid |
| 0.5% | Preservative (NIPASEPT ®) |
| 0.5% | Perfume |
| To 100% | Water |

### Example 4

### Hair Conditioning Agent (Leave-in)

| | |
|---|---|
| 2.0% | Cetearyl alcohol |
| 0.6% | Behentrimonium chloride |
| 1.0% | Quaternium-80 (ABIL ® QUAT 3272) |
| 0.5% | Phenylpropyldimethylsiloxysilicate |
| | (SILSHINE ® 151) |
| 2.0% | Propylene glycol |
| 0.5% | Perfume |
| 0.5% | Preservative (NIPASEPT ®) |
| To 100% | Water |

### Example 5

### Hair Conditioning Agent (Pump Foam, Aerosol Foam)

| | |
|---|---|
| 2.0% | Behenyl alcohol (LANETTE ® 22) |
| 8.0% | Cetrimonium chloride (QUARTAMIN ® 60W25-G) |
| 0.3% | Polyquaternium-7 (MERQUAT ® 550L) |
| 1.0% | Glycerol |
| 0.5% | Phenylpropyldimethylsiloxysilicate |
| | (SILSHINE ® 151) |
| 0.35% | Citric acid |
| 0.7% | Preservative (NIPASEPT ®) |
| 0.3% | Perfume |
| 0.2% | PARSOL ® 1789 |
| To 100% | Water |

The effective ingredient solution can be dispensed as an aerosol and also applied as foam with a hand pump.

### Example 6

### Hair Conditioning Agent (Clear)

| | |
|---|---|
| 1.5% | Hydroxyethyl cellulose (NATROSOL ® 250 HHR) |
| 2.0% | Cetrimonium chloride (GENAMIN ® CTAC 50) |
| 0.5% | Phenylpropyldimethylsiloxysilicate |
| | (SILSHINE ® 151) |
| 7.5% | Butylene glycol |
| 0.4% | Perfume |
| 0.8% | Preservative (NIPASEPT ®) |
| 0.2% | PEG-40 Hydrogenated Castor Oil |
| To 100% | Water |

### Example 7

### Conditioning Shampoo

| | |
|---|---|
| 40.0% | Sodium lauryl ether sulfate (TEXAPON ® N28) |
| 8.0% | Cocamidopropyl betaine |
| 1.0% | Cetearyl alcohol (LANETTE ® O) |
| 0.2% | Cetrimonium chloride |
| 2.0% | Phenylpropyldimethylsiloxysilicate |
| | (SILSHINE ® 151) |
| 1.5% | Propylene glycol |
| 5.0% | CARBOPOL ® AQUA SF-1 |
| q.s. | Aminoethylpropanol to pH = 6.0 |
| 0.5% | Perfume |
| 1.0% | Preservative (NIPASEPT ®) |
| To 100% | Water |

The disclosure in German Patent Application 10 2004 018 017.2-43 of Apr. 14, 2004 is incorporated here by reference. This German Patent Application describes the invention described hereinabove and claimed in the claims appended hereinbelow and provides the basis for a claim of priority for the instant invention under 35 U.S.C. 119.

While the invention has been illustrated and described as embodied in cosmetic compositions for hair care, it is not intended to be limited to the details shown, since various modifications and changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed is new and is set forth in the following appended claims.

1. A cosmetic composition with conditioning action for hair, said cosmetic composition containing
a) from 0.01 to 20.0 percent by weight of at least one siloxane polymer of formula (I): 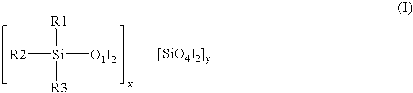
wherein R1, R2 and R3 are each, independently of each other, a linear or branched C₁- to C₆-alkyl group, phenyl group or a linear or branched phenyl-(C₁- to C₆)-alkyl group, wherein x and y represent whole numbers, x≧4 and y≧1 and y has a maximum value of 100,000 and x has a maximum value of 100,002; and
b) from 0.1 to 20.0 percent by weight of at least one polyhydric alcohol.

2. The cosmetic composition as defined in claim 1, wherein said R1, R2 and R3 each represent, independently of each other, a methyl group, a propyl group, a butyl group, a phenyl group, a phenylpropyl group or a phenylisopropyl group.

3. The cosmetic composition as defined in claim 1, wherein said at least one siloxane polymer is a phenylalkyldialkylsiloxysilicate of formula (II): 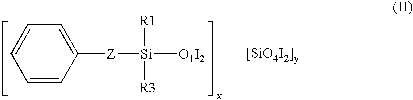
wherein Z represents a branched or unbranched C₁- to C₆-alkylene group, R1 and R2 each represent a C₁- to C₆-alkyl group, and x and y are whole numbers, x≧4 and y≧1 and y has a maximum value of 100,000 and x has a maximum value of 100,002.

4. The cosmetic composition as defined in claim 1, wherein said at least one siloxane polymer comprises a phenylpropyldimethylsiloxysilicate of formula (III): 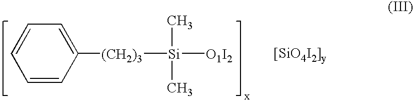
wherein x and y are whole numbers, x≧4 and y≧1 and y has a maximum value of 100,000 and x has a maximum value of 100,002.

5. The cosmetic composition as defined in claim 1, wherein said at least one polyhydric alcohol is a branched or unbranched polyhydric aliphatic alcohol with at least two hydroxyl groups.

6. The cosmetic composition as defined in claim 5, wherein said polyhydric aliphatic alcohol comprises a sugar alcohol.

7. The cosmetic composition as defined in claim 5, wherein said polyhydric aliphatic alcohol has 2 to 6 carbon atoms.

8. The cosmetic composition as defined in claim 5, wherein said polyhydric aliphatic alcohol is selected from the group consisting of ethylene glycol, 1,3-butylene glycol, 1,2-propylene glycol, propan-1,3-diol, butan-1,3-diol, caprylyl glycol, glycerol and sorbitol.

9. The cosmetic composition as defined in claim 1, further comprising 0.1 to 20.0 percent by weight of at least one fatty alcohol.

10. The cosmetic composition as defined in claim 1, further comprising 0.1 to 10.0 percent by weight of at least one cationic surfactant.

11. A method of improving feel, luster, flexibility, manageability, combability, softness and weight of hair, said method comprising applying a cosmetic composition as defined in claim 1 to the hair.
